(19) United States
(12) Patent Application Publication
(10) Pub. No.: US 2026/0061075 A1
(43) Pub. Date: Mar. 5, 2026

(54) SELECTIVE FUNCTIONAL ENHANCEMENT OF STEM CELLS AND OR THEIR GENETIC MATERIAL, WITH GENE TRANSFECTION FOR SHORT-AND LONG-TERM TREATMENT OF AGE-RELATED DISEASE STATES

(71) Applicants: Vincent C. Giampapa, Montclair, NJ (US); Elizabeth L. Parrish, Bainbridge Island, WA (US); Victor Urzola, San Rafael (CR)

(72) Inventors: Vincent C. Giampapa, Montclair, NJ (US); Elizabeth L. Parrish, Bainbridge Island, WA (US); Victor Urzola, San Rafael (CR)

(21) Appl. No.: 19/227,180

(22) Filed: Jun. 3, 2025

Related U.S. Application Data
(63) Continuation-in-part of application No. 18/982,680, filed on Dec. 16, 2024.
(60) Provisional application No. 63/690,466, filed on Sep. 4, 2024.

(51) Int. Cl. A61K 48/00; A61K 35/28; A61K 38/45; A61N 7/00; C12N 9/24; C12N 15/86

(52) U.S. Cl. CPC A61K 48/005; A61K 35/28; A61K 38/45; A61N 7/00; C12N 9/2402; C12N 15/86; C12Y 207/07049; C12N 2710/16143; C12N 2750/14143; C12Y 302/01031

(57) ABSTRACT

The function of enhancing stem cells is accomplished with a specific transfected human gene type into a stem cell of choice to allow for the regenerative potential of the selected stem cell to be greatly enhanced by combining both gene and stem cell therapies together. Specific genes for selected protein production are cultured with a stem cell of choice which then produces a supernatant composed of an exosome-gene complex. This exosome-gene complex can also be used as a therapeutic gene therapy on its own or in combination with a stem cell gene complex together for a specific cellular effect, organ or tissue of regenerative effect or general systemic result.

This application is a continuation in part of application Ser. No. 18/982,680, filed Dec. 16, 2024, which claims the benefit of application Ser. No. 63/690,466, filed Sep. 4, 2024, all of which are incorporated by reference in their entirety.

### BACKGROUND OF THE INVENTION

Stem cells have shown great promise in regenerative medicine for multiple different disease processes caused by the general effects of the cellular aging process. At the same time genetic therapies alone have also been shown to be significantly important in treatments used for specific inherited disease states as well as cancer.

The use of exosome therapy, the product of stem cells, has also been shown to have regenerative effects on both cell function and certain diseases related to the aging process.

This invention describes the concept of enhancing stem cell function with a specific manufactured human gene type which will allow for the regenerative potential of stem cells to be greatly enhanced by combining both gene and stem cell therapies together. The two primary causes of aging in humans are in loss of stem cell numbers and a decrease in gene activity. The combination of these two nascent technologies and processes, that is the replacement of stem cell numbers and the enhancement of specific gene activity in these stem cells is designed to increase health span and longevity as well as specific age-related diseases. This creates a synergistic effect for regenerative cellular therapy far beyond their individual effectiveness alone.

The use of exosomes derived macrovesicles has been successfully used by the present inventors Vincent Giampapa and John Greco in U.S. Pat. No. 10,772,911 (2020). Also, the Use of Enhancing Telomere lengths inside a human cell has been used by Vincent Giampapa and Linda Crouse of U.S. Pat. No. 10,098,922 (2018). In other pertinent part is U.S. 2014/0010801 A1 of Niedernhofer, et al regarding Composition and Methods or Restoring or Rejuvenating Stem Cells. And is U.S. 2022/0251603 A1 to Parrish, et al regarding System and Methods for Gene Therapy via Genetically Modified Viral Vectors. And yet further U.S. 2022/0325258 A1 of Kogut, et al regarding Methods for Cell and Tissue Rejuvenation; and U.S. 2022/0136011 A1 of Kalluri. And it is Telomerase-Containing Exosomes for Treatment of Age-Related Organ Dysfunction. For China Patent No. 112,695,049 A of Liu Minglu, et al.

### SUMMARY OF THE INVENTION

The invention also describes the use of specific human genes that can be used with or without exosomes. The human gene chosen can after transfection create a combination of stem cell-gene complex which results in a enhancement of the stem cell function. This therapy allows for both an increase in specific stem cell numbers as well as a enhancement of specific gene function that normally is lost and that occurs with the aging process.

The focus of this invention is to help restore enhanced cellular function and the physical functional loss occurring in humans due to the general aging process with genetically enhanced stem cells.

The functional losses of stem cells and specific gene activity in humans over time which are of main concern here include, sarcopenia and frailty, cognitive impairment, central brain regulatory mechanisms, dementia, immunosenescence, as well as the loss of cellular and general body energy production. All these effects of aging decrease quality of life and health span as well as longevity. They have been well documented in many recent peer reviewed science publications. The cellular and physical loss of functions cause trillions of dollars in global healthcare costs in the aging population. This technology may be a large benefit in decreasing the long-term care of the global aging population.

The above and yet other objects of the present invention will become apparent from the hereinafter set forth in the Detailed Description of the Invention, and Claims appended herewith.

### BRIEF DESCRIPTION OF THE DRAWINGS

FIG. 1 is a schematic view of cells, nucleus, chromosome and DNA structure.

FIG. 2 is a schematic view of the use of a viral DNA and vector binds to cell membrane and the process thereafter set forth below.

FIG. 3 is a schematic view of the aging clocks of the human body.

FIG. 4 is a cell-based direct delivery system

FIG. 5 is a schematic view of a basic structure of a MSC cell including exosomes at the surface and core proteins along with key immune and growth factors.

FIG. 6 is a schematic view of the exosomes of the MSC cell showing mRNA transcription and providing telomere ends of a mature mRNA.

### DETAILED DESCRIPTION OF THE INVENTION

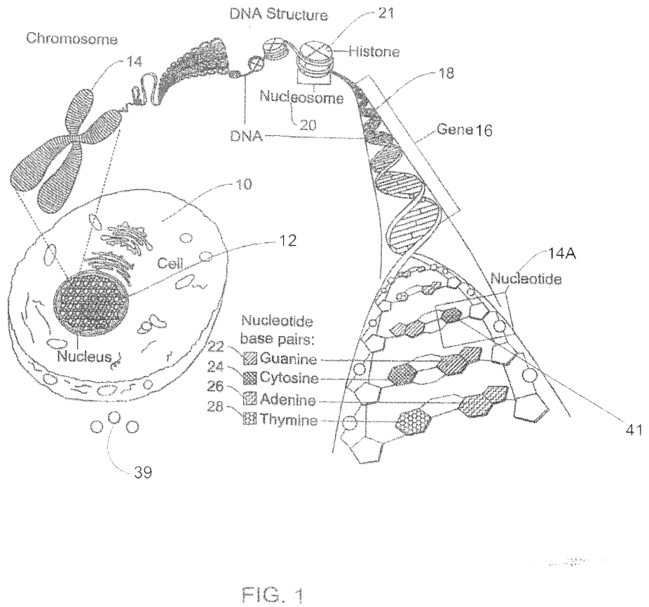

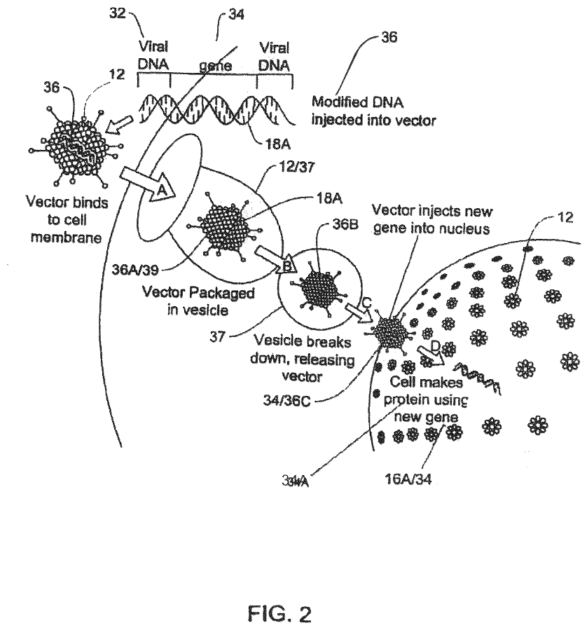

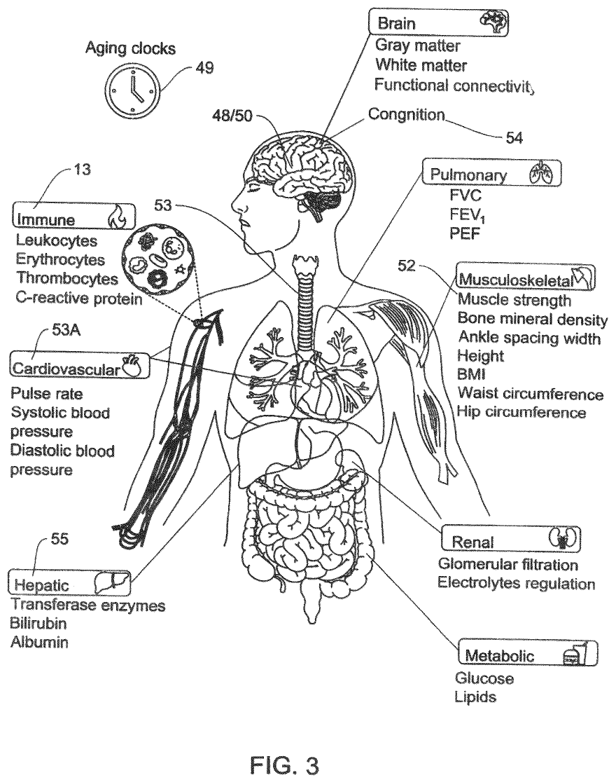

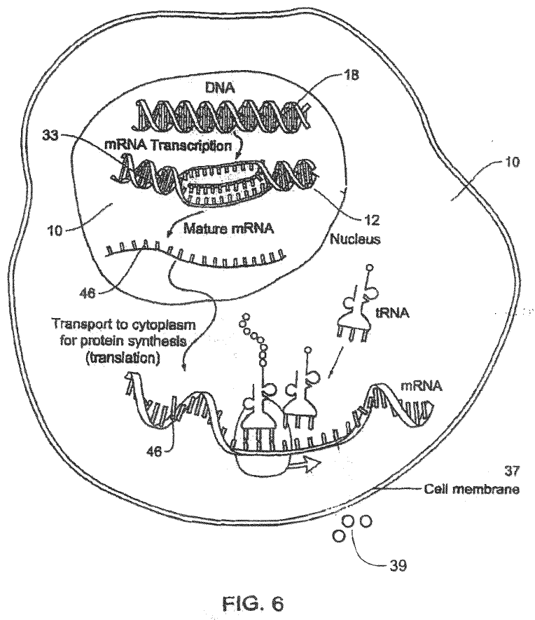

Gene vectors 32 and vector 36 (see FIG. 2) can be a AAV or CMV viral vector or plasmid 18A based, as well as other means of transfection 34 of protein 34A mentioned here. The gene 16 transferred into the nucleus 12 but does not incorporate into the original cellular genetics. It resides outside of the original genetics as “episome” or artificial micro-chromosome 14, or extra piece of DNA 18 (see FIGS. 1 and 6) which will then produce the protein of choice. The protein may leave the cell 10 and make its way into the systemic vascular system 53A of the person (see FIG. 3), or animal, treated deploying its effects throughout the body. It may also remain in the cell to alter specific functions like increased NAD and or ATP production or even induce the production of more cellular organelles like mitochondria.

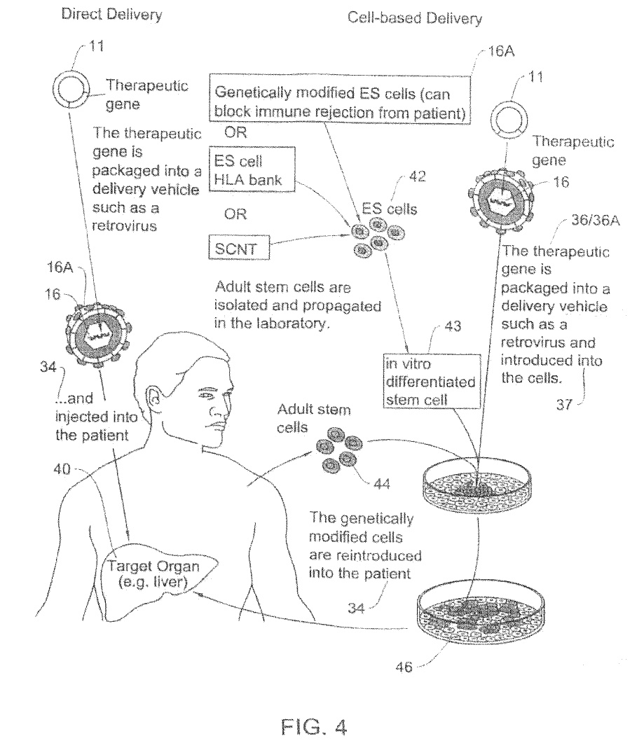

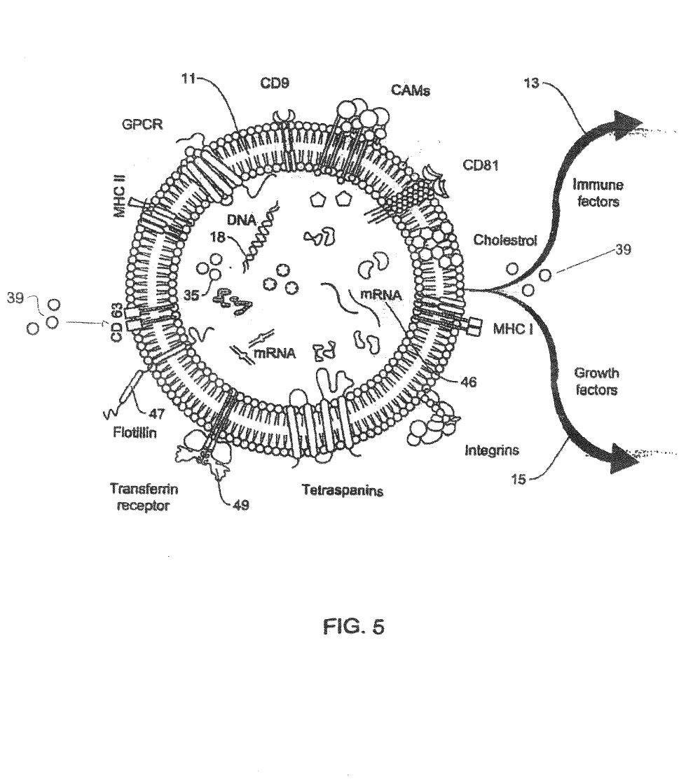

Specific genes for selected protein production may be cultured with a stem cell 11 of choice (see FIGS. 4 and 5) which will then produce a supernatant composed of an exosome-gene complex 16A/39 (see FIG. 2). This exosome-gene complex may also be used as a therapeutic gene 34 therapy on its own or in combination with the stem cell gene complex together for a specific cellular effect, organ or tissue enhancing its regenerative effect or creating general systemic effects, (see FIG. 4).

The effects of the central aging clock 49 (see FIG. 3) located in the hypothalamus 48 and the memory center in the hippocampus 50, controlling multiple aspects of the body's natural rhythms related to health and aging 54. (The circadian clock, including). The loss of neural stem cells over time in these locations with the aging process causes these centers to dysregulate and eventually become inefficient to maintain normal health and body rhythms. Restoration of the cells lining the hypothalamus 48, with stem cells that can replace the neural stem cells 44, and especially with gene enhanced stem cells 34 enhanced mesenchymal stem cells 38 (see FIG. 5) and or their exosomes 39 (see FIGS. 1, 2, 5 and 6) loaded with the example the klotho gene 41 (see FIG. 1), can rejuvenate the function of the hypothalamus 48 to reset the central aging clock 49, as well as the hippocampus 50 to enhance memory and cognition 54 normally lost with the aging process as well as other specific cognitive disease process due aging, i.e., Alzheimer DX, Parkinson DX and other age related cognitive decline processes, see FIG. 3.

Described in this patent is a gene therapy technique to treat or prevent a disease or physiological function loss, caused by the human aging process. This is accomplished by inserting a specific gene into a patient's stem cell of choice. Stem cells can be genetically modified to carry therapeutic genes 34 (FIG. 4). These modified or enhanced stem cells can then be used to regenerate damaged tissues, organs or even restore cellular function to normal or enhance these functions beyond a normal level. (see FIG. 3). The techniques described herein can be accomplished via:
1—an AAV—(attenuated adenovirus) or CMV—(cytomegalovirus) vector delivery systems 36A/39 for long-term functional enhancement (Areas A and B, see FIG. 4) or,
2—a bacterial or synthetic-based plasmid delivery system, can be used for a short-term delivery system (see FIG. 2).
   Both vector types 36 can use selected genes of choice delivered into stem cells of different cell lineage. In this case we are describing human adult stem cells, both allogeneic and autologous stem cells including but not limited to hematopoietic stem cells, endothelial progenitor cells, and mesenchymal stem cells 38 (see FIGS. 2 and 5).
   The source of these stem cells can be from peripheral blood, bone marrow or fat cells, or from umbilical cord or other humans tissue sources or induced pluripotent stem cells this can be accomplished from human sources most readily via apheresis collection after mobilization with any number of mobilizing agents including Neupogen, Mozobil and Plerixafor to obtain a combination of multiple stem cell types 42/43 (see FIG. 4).

The goal of this process is to deliver or insert a gene of choice within the nuclear compartment 12 of the stem cell, as an episome 39 (see FIGS. 2 and 5), virtually non-integrating with the original nuclear DNA 18 (see FIG. 1). This acts as an additional artificial chromosome 14 or episome 39. These transfected cells can then be used systemically or locally to enhance stem cell function and number or suppress a given protein or restore specific cellular function as in increased muscle mass 52 (see FIG. 3) or enhanced cognition 54 (see FIG. 3) as well as immune 13 enhancement along with energy production within the cell, (see FIG. 5).

The transfected cells 34 (see FIGS. 2 and 4) can then be cultured expanded to create more numbers (see Areas B and C of FIG. 2) of the enhanced cells type for specific purposes 40/55 and then reinfused intravenously and or injected into aged local tissues, injected into lymphatics or the spinal canal 53 as well as other anatomical sites and organs, (see FIG. 3).

More specifically certain stem cell lines, hematopoietic stem cells, endothelial progenitor cells and mesenchymal stem cells, can be treated with gene transfection with specific genes for a specific purpose. For example, hematopoietic stem cells, which form all cell types involved in immune system 13, can be treated with hTERT to extend their telomere length 39 (see FIG. 6) and allow them to make more copies of themselves for a much longer time than normally possible, therefore helping to restore immune function and avoid immunosenescence, which occurs to all human adults as they age. hTERT or telomerase 34/39 (see FIGS. 2 and 6) is a ribonucleoprotein polymerase that maintains telomere ends by an addition of the base pair repeat of TTAGGG 28, 26, 22 (see FIG. 1). The enzyme consists of a protein component with reverse transcriptase (hTERT) activity 16A/34 (and Area D of FIG. 2), encoded by this gene, and an RNA component 46 that serves as a template for the telomere repeat (see FIGS. 2, 5 and 6). Mesenchymal stem cells 38 (see also FIG. 5) can be treated with the flotillin gene 74, as well as PGC-1 alpha which increases the production of ATP or NAD by creating more mitochondria, any of these stem cell gene enhanced complexes can be injected either locally into muscle or given IV systemically to restore lost muscle mass and strength 52.

These same mesenchymal stem cells 38 may also be given to avoid sarcopenia and frailty that occur in all adults over time. The same mesenchymal stem cells can also be transfected with alpha klotho 41 (see FIG. 1) and injected IV along with focused ultrasound which can help guide them to the blood brain barrier and to other specific brain centers like the hypothalamus 48 and hippocampus 50 to restore the central memory and the central aging clock 49 nucleus involved in multiple physical functions. (see FIG. 3) they can also help to restore the neural stem cells 54 responsible for maintaining general homeostasis within the brain positively affecting memory as well as dementia and Parkinson disease and other cognitive diseases.

Endothelial progenitor stem cells and other precursors to blood vessel formation can be transfected with hTERT also to enhance blood vessel 49 (transferrin) repair (see FIG. 5) and create new growth angiogenesis, which is lost over time due to the aging process.

Selective non-stem cells but immune cells like natural killer cells can also be transfected with hTERT 16A to function much longer than usual, and act as a senolytic therapy to remove senescent cells, remove viral infections, as well as cancer cells and bacterial infections. This also can help to enhance immune function.

The concepts presented here are not limited to only the genes mentioned in this application but may apply to multiple other genes that can be used for different organ and tissue regeneration like the liver 55, thymus, pancreas and other organs (see FIG. 3) and tissues not mentioned here. In general, the general process described here can be used to enhance stem cell function not mentioned in this preliminary application.
The general concepts revealed here can also be applied not just to humans but to other non-human primates, monkeys and animals in general including dogs, cats, and horses. Using stem cells that naturally occur in these animals.
The intended use of this therapy is to create enhanced stem cell function on a long-term or permanent basis using different gene-based transfection technologies. Also, exosomal-gene complexes.

For therapy with or without the gene enhanced stem cells and along with these therapies and different plasmid-based gene transfection technologies. Both technologies may incorporate a start or stop gene or other technologies to control the magnitude of gene expression desired for a given purpose or amount of time if needed for a given condition or effect.

The use of exosome products 39 from the transfected stem cells that were created in the original viral vector transfection process 36/43 can also be used for therapeutic effect with or without the transfected stem cells (see FIGS. 2-4).

The two primary causes of aging in humans are loss of stem cell numbers and loss of specific health span and decreased life span. The technology described within this patent address both of these essential age-related changes, and a therapy to restore both of these deficits that occur in all humans' gene activity that causes loss of physical and mental function in humans leading to decreased health span and lifespan.

Although the present invention has been illustrated and described herein with reference to preferred embodiments and specific examples thereof, it will be readily apparent to those of ordinary skill in the art that other embodiments may perform similar functions and like results. All such equivalent embodiments and examples are within the spirit and scope of the present invention are contemplated thereby and are intended to be covered by the following claims.

1-11. (canceled)

12. A method comprising:
(a) administering one or more immunostimulants to a donor or a patient to mobilize stem cells into the bloodstream;
(b) collecting and isolating mesenchymal stem cells (MSCs) from blood of the donor or patient;
(c) generating reprogrammed stem cells (RSCs) by exposing the MSCs to one or more histone-modifying agents that alter methylation and/or acetylation patterns;
(d) producing gene-enhanced stem cells (GESs) by transfecting the RSCs with a vector, wherein the vector comprises an adeno-associated virus (AAV), a cytomegalovirus (CMV), or a plasmid DNA vector,
wherein the vector includes a gene which encodes human telomerase reverse transcriptase (hTERT) and/or telomerase RNA component (TERC);
(e) culturing the GESs to express the human telomerase reverse transcriptase (hTERT) and/or telomerase RNA component (TERC);
(f) isolating exosomes from the GESs; and
(g) administering the isolated exosomes to a patient.

13. The method of claim 12, wherein the one or more immunostimulants comprise filgrastim, plerixafor, or a combination thereof, administered to the donor or the patient.

14. The method of claim 12, wherein the step of collecting MSCs further comprises apheresis.

15. The method of claim 12, further comprising measuring a degree of telomere elongation in the GESs following the step of culturing the GESs and before the step of isolating the exosomes.

16. The method of claim 12, wherein the step of generating RSCs comprises promoting histone demethylation by delivering JMJD3 to the MSCs.

17. The method of claim 12, wherein the step of generating RSCs comprises promoting histone acetylation by treating the MSCs with a histone deacetylase (HDAC) inhibitor.

18. The method of claim 12, wherein the step of generating RSCs comprises promoting histone demethylation by delivering JMJD3 to the MSCs and promoting acetylation by treating the MSCs with an HDAC inhibitor.

19. The method of claim 12, wherein the step of producing the GESs further comprises transfecting the RSCs to express a KL gene, which encodes a klotho protein.

20. The method of claim 12, wherein the step of producing the GESs further comprises transfecting the RSCs to express a PPARGC1A gene, which encodes PGC-1α.

21. The method of claim 12, wherein the step of producing the GESs further comprises transfecting the RSCs to express a FLOT gene, which encodes a flotillin protein.

22. The method of claim 12, wherein the step of administering the isolated exosomes comprises intravenous infusion, direct tissue injection, or intranasal delivery.
